(19) United States
(12) Patent Application Publication
(10) Pub. No.: US 2023/0128912 A1
(43) Pub. Date: Apr. 27, 2023

(54) SYSTEMS AND METHODS FOR PREDICTING ALERTNESS

(71) Applicant: ResMed Sensor Technologies Limited, Dublin (IE)

(72) Inventor: Redmond Shouldice, Dublin (IE)

(21) Appl. No.: 17/801,916

(22) PCT Filed: Feb. 27, 2021
(86) PCT No.: PCT/IB2021/051652

§ 371 Date: Aug. 24, 2022

Related U.S. Application Data
(60) Provisional application No. 62/982,608, filed on Feb. 27, 2020.
(60) Provisional application No. 63/018,206, filed on Apr. 30, 2020.

(51) Int. Cl. A61B 5/00; A61M 16/00

(52) U.S. Cl. CPC A61B 5/4806; A61M 16/024; A61M 16/0003; A61M 2016/0027

(57) ABSTRACT

A method includes (i) receiving data associated with a user during a sleep session; (ii) determining an alertness level of the user using a machine learning model that takes as input the received data; and (iii) generating a response to be communicated to the user based at least in part on the determined alertness level. The data associated with the user can be received from a respiratory therapy device configured to supply pressurized air to an airway of the user by way of a user interface coupled to the respiratory therapy device via a conduit, a sensor, or both the respiratory therapy device and the sensor.

### CROSS-REFERENCE TO RELATED APPLICATIONS

This application claims the benefit of and priority to U.S. Provisional Patent Application No. 62/982,608, filed Feb. 27, 2020 and U.S. Provisional Patent Application No. 63/018,206, filed Apr. 30, 2020, each of which is hereby incorporated by reference herein in its entirety.

### TECHNICAL FIELD

The present disclosure relates generally to systems and methods for predicting alertness, and more particularly, to systems and methods that relate alertness to a user's sleep.

### BACKGROUND

Many individuals suffer from sleep-related and/or respiratory disorders (e.g., insomnia, periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), etc.). An individual suffering from one or more of these sleep related disorders can treat or manage these disorders by modifying their behavior, activities, and/or environmental parameters (e.g., bed time, activity level, diet, etc.). Thus, it would be advantageous to develop metrics that capture effects of sleep on the individual. It would be further advantageous to develop approaches to calculating these metrics and communicating the calculated results or any information gleaned from the calculated results to the individual to aid in reducing the one or more sleep-related disorders. The present disclosure is directed to solving these and other problems.

### SUMMARY

According to some implementations of the present disclosure, a method includes (i) receiving data associated with an individual during a sleep session; (ii) determining an alertness level of the individual using a machine learning model that takes as input the received data; and (iii) generating a response to be communicated to the individual based at least in part on the determined alertness level.

According to some implementations of the present disclosure, a method for predicting an alertness level of a target user includes receiving historical sleep-session data associated with at least one person for a plurality of historical sleep sessions. The at least one person has used a respiratory therapy system during the plurality of historical sleep sessions. Historical alertness data associated with the at least one person is received. The alertness data includes alertness levels associated with each of the at least one person outside the plurality of historical sleep sessions. A machine learning model is trained with the received historical sleep-session data and the received historical alertness data such that the machine learning model is configured to (i) receive as input a current sleep-session data associated with the target user and (ii) determine as an output a predicted alertness level associated with the target user at one or more points in time.

According to some implementations of the present disclosure a method includes receiving data associated with a user during a sleep session. The user is associated with a respiratory therapy device configured to supply pressurized air to an airway of the user by way of a user interface coupled to the respiratory therapy device via a conduit. An alertness level of the user is determined using a machine learning model that takes as input the received data. A response to be communicated to the user is generated based at least in part on the determined alertness level.

According to some implementations of the present disclosure, a method includes causing a test to begin. The test includes causing a stimulus to be generated at a first point in time. A response to the stimulus is received from a user at a second point in time. The response includes an expelled air current from the user that is detected using a respiratory therapy device configured to supply pressurized air to an airway of the user. The pressurized air is supplied by way of a user interface coupled to the respiratory therapy device via a conduit. A first score is determined based at least in part on (i) the first point in time and (ii) the second point in time.

According to some implementations of the present disclosure, a method includes causing a test to begin. The test includes causing a stimulus to be generated at a first point in time. A response to the stimulus is received from a user at a second point in time. The response is detected using a respiratory therapy system including a respiratory therapy device, a conduit, and a user interface. The respiratory therapy device is configured to supply pressurized air to an airway of the user by way of the user interface that is coupled to the respiratory therapy device via the conduit. A first score is determined based at least in part on (i) the first point in time and (ii) the second point in time.

According to some implementations of the present disclosure, a method includes: (a) causing a first test to begin, the first test including causing a first stimulus to be generated at a first point in time; (b) receiving a first response to the first stimulus from a user at a second point in time, the first response being detected using a respiratory therapy system including a respiratory therapy device, a conduit, and a user interface, the respiratory therapy device being configured to supply pressurized air to an airway of the user by way of the user interface that is coupled to the respiratory therapy device via the conduit; (c) determining a first score based at least in part on (i) the first point in time and (ii) the second point in time; (d) causing the respiratory therapy device to deliver the supplied pressurized air to the user during a first therapy session; (e) causing a second test to begin, the second test including causing a second stimulus to be generated at a third point in time; (f) receiving a second response to the second stimulus from the user at a fourth point in time, the second response being detected using the respiratory therapy system; (g) determining a second score based at least in part on (i) the third point in time and (ii) the fourth point in time; and (h) communicating a result associated with the first score and the second score to the user.

According to some implementations of the present disclosure, a system includes a respiratory device configured to supply pressurized air to an airway of a user, the pressurized air being supplied by way of a user interface coupled to the respiratory device via a conduit. The system further includes a memory storing machine-readable instructions. The system further includes a control system including one or more processors configured to execute the machine-readable instructions to: (a) cause a test to begin, the test including causing a stimulus to be generated at a first point in time; (b) receive a response to the stimulus from the user at a second point in time, the response including an expelled air current from the user that is detected using the respiratory device; and (c) determine a first score based at least in part on (i) the first point in time and (ii) the second point in time.

According to some implementations of the present disclosure, a system includes a respiratory system including a respiratory device, a conduit, and a user interface. The respiratory device is configured to supply pressurized air to an airway of a user by way of the user interface that is coupled to the respiratory device via the conduit. The system further includes a memory storing machine-readable instructions. The system further includes a control system including one or more processors configured to execute the machine-readable instructions to: (a) cause a test to begin, the test including causing a stimulus to be generated at a first point in time; (b) receive a response to the stimulus from the user at a second point in time, the response being detected using the respiratory system; and (c) determine a first score based at least in part on (i) the first point in time and (ii) the second point in time.

According to some implementations of the present disclosure, a system includes a respiratory system including a respiratory device, a conduit, and a user interface. The respiratory device is configured to supply pressurized air to an airway of a user by way of the user interface that is coupled to the respiratory device via the conduit. The system further includes a memory storing machine-readable instructions. The system further includes a control system including one or more processors configured to execute the machine-readable instructions to: (a) cause a first test to begin, the first test including causing a first stimulus to be generated at a first point in time; (b) receive a first response to the first stimulus from the user at a second point in time, the first response being detected using the respiratory system; (c) determine a first score based at least in part on (i) the first point in time and (ii) the second point in time; (d) cause the respiratory device to deliver the supplied pressurized air to the user during a first therapy session; (e) cause a second test to begin, the second test including causing a second stimulus to be generated at a third point in time; (f) receive a second response to the second stimulus from the user at a fourth point in time, the second response being detected using the respiratory system; (g) determine a second score based at least in part on (i) the third point in time and (ii) the fourth point in time; and (h) communicate a result associated with the first score and the second score to the user.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 is a flow diagram for performing a reaction time test, according to some implementations of the present disclosure;

FIG. 5 is a flow diagram for comparing two reaction time tests, according to some implementations of the present disclosure;

FIG. 6A illustrates a user wearing a user interface with a light source, according to some implementations of the present disclosure;

FIG. 6B illustrates the user of FIG. 6A receiving a light stimulus via the light source, according to some implementations of the present disclosure;

FIG. 6C illustrates the user of FIG. 6A responding to the light stimulus, according to some implementations of the present disclosure;

FIG. 7 is a flow diagram for generating a response for a user based on alertness, according to some implementations of the present disclosure; and

FIG. 8 is a flow diagram for training a machine learning model to predict an alertness level of a target user, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood. Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness. Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping. To help the individual sleep better and reduce any of the aforementioned events, a physician can prescribe or recommend the use of respiratory therapy systems. Patients starting to use a respiratory therapy system for the first time may not feel an immediate health benefit of using the respiratory therapy system. This lack of an immediate subjective health benefit can cause some patients to stop using the respiratory therapy system or to prematurely conclude that the respiratory therapy system is ineffective. Although subjective improvements may be hard to gauge after a first therapy session on the respiratory therapy system, objective measures or metrics can show improvement. Without objective measures or metrics, some patients may stop using the respiratory therapy system, hence, some implementations of the present disclosure provide patients with information including objective measures for encouraging the patients to adhere to the prescribed or recommended use of respiratory therapy systems.

The objective measures or metrics can be obtained either passively or can require active user engagement. An example of a passively obtaining an objective measure includes obtaining heart rate variability information for a patient or obtaining heart rate data for the patient using a fitness tracker over a period of time. The fitness tracker can passively obtain heart rate data without the patient engaging with the fitness tracker. An example of an active user engagement is having the patient perform one or more tests, which tests are indicative of the quality of sleep had by the user prior to the tests. The tests can be reaction time tests where a reaction time of the patient is measured. The tests can be sustained attention, reaction time test where the patient is engaged for a longer period of time compared to a simple reaction time test. An example of a sustained attention, reaction time test includes a psychomotor vigilance task (PVT) test. Some implementations of the present disclosure will be described in connection with a reaction time test merely for illustration purposes. The implementations of the present disclosure are not limited to only reaction time tests.

Some implementations of the present disclosure provide objective measures of vigilance before and after a recommended or prescribed therapy with a respiratory therapy system. Vigilance is related to alertness and watchfulness. Untreated OSA can lead to daytime sleepiness and concomitant reduced alertness. As such, slower responses captured using tests can indicate sleepiness or in some cases lack of attention or engagement.

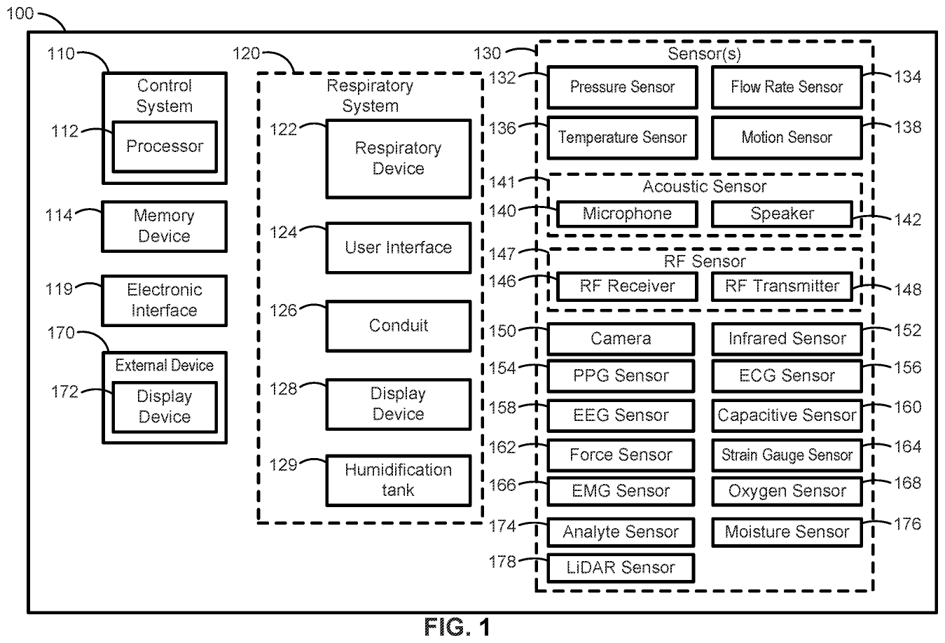

Referring to FIG. 1, a system 100, according to some implementations of the present disclosure, is illustrated. The system 100 includes a control system 110, a memory device 114, an electronic interface 119, one or more sensors 130, a respiratory therapy system 120, and one or more user devices 170.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, a housing of one or more of the sensors 130, and/or a housing of a respiratory therapy device 122 included in the respiratory therapy system 120. The control system 110 can be centralized (within one such housing) or decentralized (within two or more of such housings, which are physically distinct). In such implementations including two or more housings containing the control system 110, such housings can be located proximately and/or remotely from each other. One or more of the sensors 130 can be coupled to an external device that is distinct and separate from the respiratory therapy device 122. For example, one or more of the sensors 130 can be included in jewelry like a ring, a necklace, etc. One or more of the sensors 130 can be provided on or within a housing of the respiratory therapy device 122.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

The electronic interface 119 is configured to receive data (e.g., physiological data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

The respiratory therapy system 120 can include a respiratory pressure therapy device (e.g., the respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

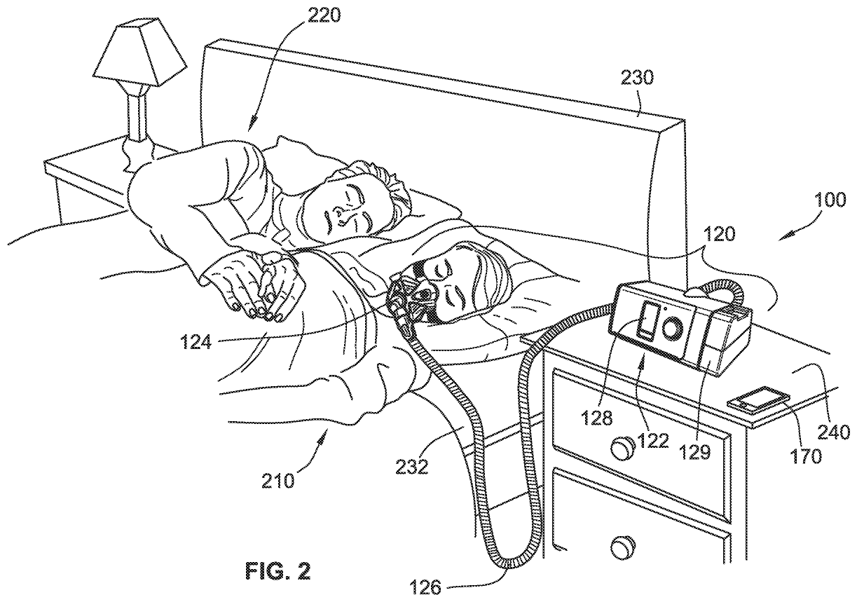

As shown in FIG. 2, in some implementations, the user interface 124 is a facial mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps (e.g., including hook and loop fasteners) for positioning and/or stabilizing the user interface 124 on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score (also referred to as a myAir™ score, such as described in WO 2016/061629, which is hereby incorporated by reference herein in its entirety), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory therapy system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, a thermal imaging sensor, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The physiological data generated by one or more of the sensors 130 can be used by the control system 110 to determine a sleep-wake signal associated with a user during a sleep session and one or more sleep-related parameters. The sleep-wake signal can be indicative of one or more sleep states, including wakefulness, relaxed wakefulness, micro-awakenings, sleep stages such as a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to determine a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Methods for determining sleep states and/or sleep stages from physiological data generated by one or more of the sensors, such as the sensors 130, are described in, for example, WO 2014/047310, U.S. 2014/0088373, WO 2017/132726, WO 2019/122413, and WO 2019/122414, each of which is hereby incorporated by reference herein in its entirety. A sleep stage could be discrete value, or a continuous variable that represents find grained variation between states—e.g., a transition from REM to Slow Wave Sleep (SWS) could include an arbitrary number of levels, as would the depth or shallowness of REM, N3 SWS, light sleep N1, N2 etc.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The microphone 140 outputs sound data that can be stored in the memory device 114 and/or analyzed by the processor 112 of the control system 110. The microphone 140 can be used to record sound(s) during a sleep session (e.g., sounds from the user 210) to determine (e.g., using the control system 110) one or more sleep-related parameters, as described in further detail herein. The microphone 140 can be coupled to or integrated in the respiratory therapy device 122, the use interface 124, the conduit 126, or the user device 170. The microphone 140 can be combined with other microphones, and beam forming can be performed. In some implementations, the one or more microphones 140 are electrically connected with a circuit board of the respiratory therapy device 122, which may be in acoustic communication (for example, via a small duct and/or a silicone window as in a stethoscope) or in fluid communication with the airflow in the respiratory therapy system 120. The microphone(s) can be incorporated an external device such as a smart device, an Internet of Things (IoT) device, a smart speaker, a smart display, a phone, a tablet, a watch, a ring, a patch, a pendant, a security camera, a security sensor, or any combination thereof. In some cases, some of the sensors can be a vehicle, such as a car, to monitor alertness.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). The speaker 142 can be coupled to or integrated in the respiratory therapy device 122, the user interface 124, the conduit 126, or the external device 170.

The microphone 140 and the speaker 142 can be used as separate devices. In some implementations, the microphone 140 and the speaker 142 can be combined into an acoustic sensor 141 (e.g. a sonar sensor), as described in, for example, International (PCT) Publication number WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory device 122, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a radar sensor). In some such implementations, the RF sensor 147 includes a control circuit. The specific format of the RF communication could be WiFi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine some of the one or more parameters discussed above in connection with the PPG sensor 154.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state and/or sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth. The mask could also be part of a head-mounted (head-worn) PAP system, whereby the full respiratory therapy system is worn on the head, and comprising integrated (or external) sensors, such as described elsewhere in this document. The sensors could also be integrated into a “smart mask,” with a separate respiratory therapy device and control circuitry.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The user device 170 (FIG. 1) includes a display device 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch, a fitness tracker, etc.). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for generating physiological data and determining a recommended notification or action for the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems can be formed using any portion or portions of the components shown and described herein and/or in combination with one or more other components.

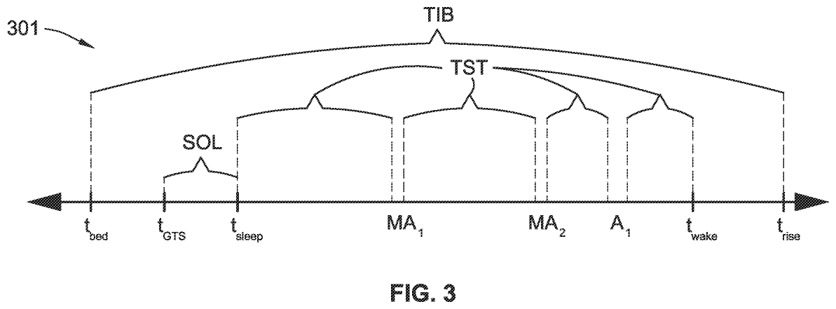

As used herein, a sleep session can be defined in a number of ways based on, for example, an initial start time and an end time. Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁ and a second micro-awakening MA₂, a wake-up time (t_{wake}), and a rising time (tᵣᵢₛₑ).

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

Referring back to FIG. 3, the enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (t_{GTS}) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings there-between. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

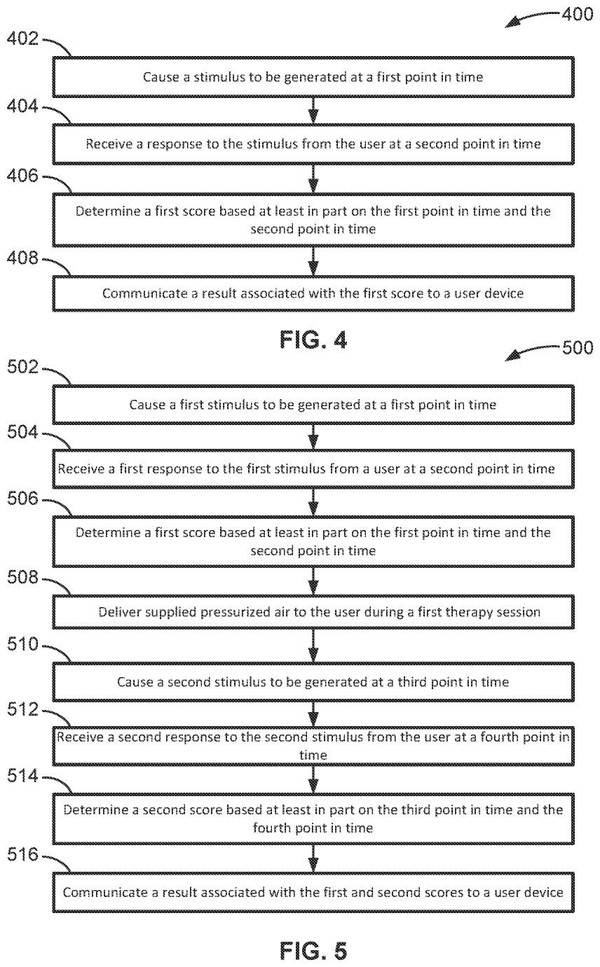

Referring to FIG. 4, a method 400 for performing a reaction time test is illustrated according to some implementations of the present disclosure. One or more steps of the method 400 can be implemented using any element or aspect of the system 100 (FIGS. 1-2) described herein.

Step 402 of the method 400 includes causing a reaction time test to begin by causing a stimulus to be generated at a first point in time. In some implementations, the stimulus can be generated by, for example, a light source. The light source can be a light emitting diode (LED) coupled to the respiratory therapy device 122, the electronic device 170, the user interface 124, the conduit 126, or any combination thereof. In some implementations, the display device 128 of the respiratory therapy system 120 and/or the display device 172 of the user device 170 can be used as the light source to generate the stimulus.

In some implementations, the stimulus can be sound generated by a speaker, for example, the speaker 142 or a speaker coupled to a housing of the user device 170. For example, the user device 170 can be a smart speaker, and the smart speaker can generate the sound stimulus. In some implementations, the speaker generating the sound stimulus is at least partially positioned within a housing of the respiratory therapy device 122 and/or coupled to the user interface 124.

In some implementations, the stimulus can be vibration generated by and/or caused by a motor of the respiratory therapy device 122. For example, the rotations per minute (RPM) of the motor of the respiratory therapy device 122 can be increased and/or decreased to cause the respiratory therapy device 122 to vibrate. For example, the RPMs can be changed from 3000 RPMs to 10,000 RPMs to cause vibration in some implementations. The RPM of the motor can oscillate between two or more different levels. The user 210 (FIG. 2) can interpret the sudden vibration of the motor as an indication of receiving a vibratory stimulus. The vibratory stimulus can come from other sources, for example, a vibration of the user device 170. For example, a smart phone or an alarm clock of the user 210 can vibrate, providing the user 210 with the vibratory stimulus.

In some implementations, the stimulus can be generated by varying a pressure of the generated pressurized air supplied by the respiratory therapy device 122. For example, the user 210 dons the user interface 124, and the respiratory therapy device 122 provides pressurized air to the user 210 at a first pressure level. The respiratory therapy device 122 can increase or decrease pressure of the pressurized air to a second pressure level, thus providing the stimulus to the user 210. The second pressure level is different from the first pressure level in a manner that the user 210 can sense the change in pressurized air being provided by the respiratory therapy device 122. In some implementations, the respiratory therapy device 122 returns the pressure of the pressurized air from the second pressure level back to the first pressure level. That way, the generated stimulus includes pressurized air being provided at either a temporarily increased pressure level or a temporarily decreased pressure level that interrupts the first pressure level for a period of time.

In some implementations, the stimulus includes the respiratory therapy device 122 stopping supply of pressurized air to the user 210. In some implementations, the stimulus includes the respiratory therapy device 122 starting supply of pressurized air to the user 210.

Step 404 of the method 400 includes receiving a response to the stimulus from a user 210 at a second point in time. In some implementations, the response is an expelled air current from the user 210 that is detected using the system 100. For example, the flow rate sensor 134 and/or the pressure sensor 132 can be coupled to the respiratory therapy device 122, the user interface 124, the conduit 126, or any combination thereof. The flow rate sensor 134 can detect the expelled air current as a change in flow rate within the conduit 126. Similarly, the pressure sensor 132 can detect the expelled air current as a change in pressure within the conduit 126.

In some implementations, the expelled air current from the user 210 is detected using the microphone 140. The microphone 140 can capture a breathing pattern of the user 210, and based on a timing of the generated stimulus of step 402 and the expelled air current being detected by the microphone 140, the control system 110 can determine that the expelled air current being detected by the microphone 140 is in response to the generated stimulus. For example, the control system 110 can determine that the expelled air current does not fit the breathing pattern of the user 210, and as such must be in response to the generated stimulus of step 402.

In some implementations where the expelled air current from the user 210 is being detected as the response to the generated stimulus of step 402, a timing of the generated stimulus is correlated with a breathing pattern of the user 210. For example, when the control system 110 determines that the user 210 is about to begin breathing out after taking an in-breath, then the control system 110 does not generate the stimulus. The control system 110 can provide the stimulus such that the stimulus is generated when the user 210 is about to start an in-breath or anytime during the user 210 breathing in. The control system 110 can analyze the breathing pattern of the user 210 under these scenarios to detect departures from the breathing pattern to determine whether the user 210 is responding to the generated stimulus via an expelled air current. That is, sudden increases in out-breath pressure or cutting short an in-breath and switching to an out-breath can be indicative of responding to the generated stimulus with an expelled air current. In some implementations, a correction factor can be included in view of a delay, associated with when, within the breathing cycle (or the breathing pattern) of the user 210, the stimulus is provided to the user 210.

In some implementations, the breathing cycle of the user 210 can fit a rhythm can be expressed as a sinusoidal graph. An apex of the sinusoidal graph represents a moment where the user 210 breathes in completely within the breathing cycle, and a trough of the sinusoidal graph represents a moment where the user 210 breathes out completely within the breathing cycle. If the stimulus is provided to the user 210 at a trough, just as the user 210 is beginning to breathe in, the user 210 may experience difficulties expelling air without first continuing to breathe in, in order to have air to expel. On the other hand, if the stimulus is provided to the user 210 at an apex, just as the user is beginning to breathe out, the user 210 may easily expel air. As such, a magnitude of the correction factor may be greater when the stimulus is provided at a trough of the breathing cycle when compared to when the stimulus is provided at an apex of the breathing cycle.

In some implementations, the user 210 can breathe normally using the respiratory therapy system 120, and prior to providing the at step 402, a pressure and/or flow rate of pressurized air supplied to the user 210 using the respiratory therapy system 120 is monitored. The pressure and/or flow rate can be monitored for five seconds, ten seconds, twenty seconds, etc. Monitoring the pressure and/or flow rate allows the control system 110 to determine variations in pressure and/or flow rate due to a normal breathing pattern of the user 210. Determining variations in pressure and/or flow rate due to a normal breathing pattern of the user 210 allows the control system 110 to detect a departure from the normal breathing pattern as a response to the stimuli.

In some implementations, at step 404, the response from the user 210 is a tap, a touch, or more generally, a purposeful physical contact with or movement of the user device 170, the respiratory therapy device 122, the user interface 124, and/or the conduit 126. The purposeful physical contact can be detected by the display device 172 of the user device 170 or the display device 128 of the respiratory therapy system 120 (e.g., the display device 172 or the display device 128 is a touch sensitive display configured to receive touch inputs from the user 210). The sensed purposeful physical contact can be due to a movement of the head of the user 210.

In some implementations, the purposeful physical contact can be detected by the one or more sensors 130. For example, the conduit 126 or the user interface 124 can include the force sensor 162 for detecting an external force or disturbance in either the conduit 126 or the user interface 124. For example, the force sensor 162 can include an accelerometer that detects movement of the conduit 126 or the user interface 124. In another example, the strain gauge sensor 164 or the capacitive sensor 160 can be included on the user interface 124 for detecting a tap or touch on the user interface 124. The one or more sensors 130 can be used to determine whether the user 210 shifts position in response to the generated stimuli of step 402, whether the user 210 moves her head in response to the generated stimuli, or whether the user 210 makes a gesture that disturbs or is detected at the user interface 124 or the conduit 126. In some implementations, the purposeful physical contact can include pressing a button coupled to user interface 124.

In some implementation, the response from the user 210 includes gestures that may not be classified as purposeful physical contact. The gestures can include hand movement (e.g., a wave, a thumbs up, a peace sign, etc.), head movement (e.g., a nod, a shake of the head, etc.), facial movements (e.g., a smile, raised eyebrows, etc.). The gestures can be detected by the one or more sensors 130 integrated in a smart device and/or in the respiratory therapy system 120. For example, the motion sensor 138, the IR sensor 152 and/or the camera 150 can be used to capture video and/or sequential images to detect the gestures.

In some implementations, the response from the user 210 is verified by multiple sensors of the one or more sensors 130. For example, any of the motion sensor 138, the acoustic sensor 141, the RF sensor 147, or a combination thereof can be used by the control system 110 to increase confidence in the purposeful physical contact and/or non-physical contact gestures detected by the one or more sensors 130. For example, the motion sensor 138 can sense a movement of the user 210 before a tap is detected by the force sensor 162 on the user interface 124 or on the conduit 126. Combining the movement sensed by the motion sensor 138 with that of the force sensor 162 increases confidence that the user 210 responded to the generated stimulus of step 402. In some implementations, audio from the microphone 140 can be combined with image captures from the camera 150 and/or the IR sensor 152 to verify a non-physical contact gesture.

In some implementations, at step 404, the response from the user 210 is voice of the user 210 captured by the microphone 140. The microphone 140 can include an array of microphones such that the voice captured by the microphone 140 can be localized. That way, other sensors within the one or more sensors 130 can be used to verify that the voice captured by the microphone 140 is coming from the user 210 and not someone else (e.g., the bed partner 220).

Step 406 of the method 400 includes determining a first score based at least in part on the first point in time and the second point in time. In some implementations, the control system 110 generates a first timestamp for when the generated stimulus of step 402 was presented to the user 210 and a second timestamp for when the response of step 404 was received from the user 210. The control system 110 can then calculate the first score to be a difference between the first timestamp and the second timestamp.

In some implementations, the difference between the first timestamp and the second timestamp is adjusted for different variables. For example, one or more delays associated with mechanics of the response of the user 210 is taken into account. In an example, the user 210 can see an LED light up on the user interface 124 and can quickly tap on the user interface 124 to register a response. Depending on where the palm of the user 210 is located, the user 210 can either have a long delay before her palm reaches the user interface 124 or a short delay before her palm reaches the user interface 124. This delay can be taken into account when determining the first score.

In another example, the user 210 can be at the beginning of an in-breath when the stimulus is provided and as such, the user 210 determines to draw enough breath to reach a minimum lung capacity before expelling the air from her lungs as the response of step 404. The delay in reaching the minimum lung capacity comfortable enough for the patient to expel the air can be taken into account when determining the first score.

In another example, delay dynamics related to generation of a light stimulus or generation of a sound stimulus are taken into account. For example, a finite delay can exist between the control system 110 determining that the light stimulus be provided to when a light source actually provides the light signal. Similarly, a finite delay can exist between the control system 110 determining that a sound stimulus be provided to when the sound stimulus is actually generated. These delay dynamics can be used to determine correction factors for when determining the first score.

In some implementations, noise effects are taken into account when determining the first score. For example, bouncing effects like switch bounces or multiple successive registering of the received response of step 404 is smoothed out with a high pass signal filter.

In some implementations, steps 402 and 404 are repeated multiple times to obtain multiple pairs of timestamps for each stimulus-response pair. The multiple pairs of timestamps can be used in various statistical models to determine the first score. For example, for each of the multiple pairs of timestamps, a duration between each pair of timestamps is calculated. Each of the durations can be included in a set for determining a median, a mode, a mean, or a weighted mean as the first score. In some implementations, prior to applying statistical models to determine the first score, each of the durations is adjusted for different variables as previously discussed.

In some implementations, steps 402 and 404 are repeated multiple times to obtain the multiple pairs of timestamps over a period of time. The period of time can be at least thirty seconds. In some instances, the user 210 fails to respond within a threshold period (e.g., fails to respond within 0.5 seconds), and the control system 110 determines that the generated stimulus was ignored by the user 210 and does not include the timestamp of the ignored stimulus when determining the first score. In some implementations, steps 402 and 404 are only performed once over the period of time.

In some implementations, steps 402 and 404 are repeated multiple times when the response at step 404 is an expelled air current from the user 210. Each of the stimuli generated at step 402 is provided at different points during a breathing pattern of the user 210. For example, a stimulus can be generated multiple times during an in-breath, an out-breath, or a combination of both. Receiving responses for each of the generated stimuli provides data for multiple stimulus-response pairs. The data for the multiple stimulus-response pairs can be averaged, regressed, or filtered to mitigate effects associated with catching the user 210 with a stimulus at an inopportune time (e.g., providing the user 210 with a stimulus at the end of an out-breath and forcing the user to quickly breath in to respond to the stimulus).

In some implementations, the first score is determined based on receiving user feedback provided by the user 210. The user feedback can include an analog rating of a subjective energy level of the user 210. The user device 170 (e.g., via alphanumeric text, speech-to-text, etc.) can receive the user feedback. In some implementations, the user 210 is prompted to provide the user feedback. For example, the control system 110 can cause one or more prompts to be displayed on the display device 172 of the user device 170 that provides an interface for the user 210 to provide the user feedback (e.g., the user clicks or taps to enter feedback, the user enters feedback using an alphanumeric keyboard, etc.). The received user feedback can be stored, for example, in the memory device 114.

Optionally, step 408 of the method 400 includes communicating a result associated with the first score to the user, such as via user device 170. For example, the communicated result can include the first score along with data collected for generating the first score. The data collected can be provided in a comma-separated values file such that the user device 170 can graph the data collected during the reaction time test. The result is communicated to the user, such as via user device 170, to help the user 210 gauge a baseline performance for the reaction time test.

In some implementations, the method 400 is performed multiple times throughout a daily or weekly schedule of the user 210. For example, the user 210 wakes up in the morning still being coupled to the user interface 124. The control system 110 determines a sleep-wake signal associated with the user 210 that indicates that the user 210 has changed sleep states into a wakefulness sleep state. The control system 110 can then begin a first test by causing the stimulus at step 402 to be generated, receiving the response at step 404 using the respiratory therapy system 120, and determining the first score. During the day, the control system 110 can cause the user device 170 to begin a second test such that the user 210 engages with the user device 170 to determine a second score. In some implementations, the second test is performed based on a time of day, for example, during a lunch break of the user 210. Prior to going to bed, the user 210 can engage with the respiratory therapy system 120, and the control system 110 can cause the respiratory therapy system 120 to perform a third test to obtain a third score.

In some implementations, prior to generating the stimulus at step 402, the control system 110 determines whether the user 210 is prepared to receive the stimulus. The control system 110 can provide an alert to the user 210 informing the user of when the reaction time test illustrated by the method 400 will begin, such as an alert that the reaction time test will begin within an indicated period of time, e.g. within 30-60 seconds or 1-2 minutes of the alert. The alert provides the user 210 a forewarning to prevent the user 210 from disengaging from the user interface 124 or from performing another task that may distract the user 210. The alert can include a prompt displayed on the display device 172 of the user device 170 or the display device 128 of the respiratory therapy system 120. The alert can include sound emitted by a speaker partially positioned within a housing of the respiratory therapy device 122 or a speaker coupled to a housing of the user device 170. The alert can include noise generated by a motor of the respiratory therapy device 122 or a motor of the user device 170. The alert can include light generated by a light source coupled to the respiratory therapy system 120 or a light source coupled to the user device 170. The alert can include vibration of the motor of the respiratory therapy device 122 or the motor of the user device 170.

In some implementations, the user 210 provides an acknowledgment of the provided alert. The acknowledgment indicates that the user 210 is ready to begin the reaction time test. The acknowledgement can include a touch signal generated by the display device 172 or the display device 128 in response to the user 210 touching the display device 172 or the display device 128, respectively. The acknowledgment can include a press signal on a button coupled to the housing of the respiratory therapy device 122 or from a button coupled to a housing of the user device 170. The acknowledgment can include voice data received via the microphone 140.

In some implementations, when the method 400 is performed using different systems (e.g., using the user device 170 to generate the stimulus and receive the response, using the respiratory therapy system 120 to generate the stimulus and receive the response, etc.), the control system 110 is configured to determine a normalized relationship for relating scores obtained from one system with scores obtained from another system. For example, a touch input as a response on the respiratory therapy system 120 or the user device 170 can be quicker to detect than detecting an expelled air current of the user 210. Thus, when determining scores throughout the day using various stimuli and various responses, the determined scores can be normalized to remove discrepancies associated with modes of obtaining responses from the user 210.

In some implementations, the control system 110 is configured to determine the normalized relationship over a period of time. The control system 110 can track and associate each of the determined scores with a respective mode of obtaining responses from the user 210. That way, prior to performing any reaction time tests, the control system 110 can predict a score for a specific mode of obtaining a response. The predicted score can be used to gauge an accuracy of the determined normalized relationship. As more scores are determined from each mode, the normalized relationships become more accurate. In some implementations, the predicted scores being within a percentage (e.g., 1%, 2%, 5%, etc.) of the determined score is deemed accurate.

Referring to FIG. 5, a method 500 according to some implementations of the present disclosure is illustrated. The method 500 can be implemented using any combination of elements or aspects of the system 100 described herein.

Step 502 of the method 500 is the same as, or similar to step 402 of the method 400 (FIG. 4) and includes causing a first stimulus to be generated at a first point in time. Generation of the first stimulus can indicate a beginning of a first test.

Step 504 of the method 500 is the same as, or similar to, step 404 of the method 400 (FIG. 4) and includes receiving a first response to the first stimulus from a user (e.g., the user 210 of FIG. 2) at a second point in time. The first response to the first stimulus can be detected using the respiratory therapy system 120.

Step 506 of the method 500 is the same as, or similar to, step 406 of the method 400 (FIG. 4) and includes determining a first score based at least in part on the first point in time and the second point in time. One or more of steps 502, 504, or 506 can be performed multiple times.

Step 508 of the method 500 includes delivering pressurized air to the user 210 during a first sleep session. For example, the first score is determined prior to the user 210 going to bed. While sleeping, the respiratory therapy system 120 delivers pressurized air to the user 210 as depicted in FIG. 2.

Step 510 of the method 500 is the same as, or similar to step 402 of the method 400 (FIG. 4) and includes causing a second stimulus to be generated at a third point in time. Generation of the second stimulus can indicate a beginning of a second test. Step 510 is performed after the first sleep session while the user 210 is awake.

Step 512 of the method 500 is the same as, or similar to, step 404 of the method 400 (FIG. 4) and includes receiving a second response to the second stimulus from the user 210 at a fourth point in time. The second response to the second stimulus can be detected using the respiratory therapy system 120.

Step 514 of the method 500 is the same as, or similar to, step 406 of the method 400 (FIG. 4) and includes determining a second score based at least in part on the third point in time and the fourth point in time. One or more of steps 510, 512, or 514 can be performed multiple times.

Step 516 of the method 500 is similar to step 408 of the method 400 (FIG. 4) and includes communicating a result associated with the first and the second scores to the user, such as via user device 170. The result can include statistical analyses between the first and the second scores.

In some implementations, the result communicated to the user device 170 can include physiological data (e.g., heart rate, breathing rate, EEG, EMG, electrooculography (EOG), pupillography, etc.), and/or demographic data (e.g., age, gender, etc.). The one or more sensors 130 can gather physiological data of the user 210 prior to or during the first test and physiological data of the user 210 after or during the second test. The physiological data can provide additional insights regarding therapy with the respiratory therapy system 120. For example, resting heart rate and heart rate variability (HRV) are expected to drop over time with sustained use of the respiratory therapy system 120.

In some implementations, the gathered physiological data of the user 210 and/or demographic data can be used to make inferences or predictions. For example, a change in heart rate, such as in HRV, can be captured during reaction time tests (e.g., during the first test, the second test, and any other subsequent tests) at different times. The captured HRV can be used as (or can be used to determine) reference values of typical changes in heart rate that relate to a specific level of vigilance state (alert, drowsy etc.). As such, gathered physiological data of the user 210 can be correlated with reaction times such that future monitored changes in HRV can be related to an estimated reaction time and/or can be related to an efficacy of therapy. In some implementations, depending on the mode of the stimulus used in the tests (e.g., auditory stimulus, light stimulus, pressure change, etc.) and the mode of the response in the tests, the tests may be categorized. The categorization allows different correction of calibration factors across the different modes.

In some implementations, the control system 110 can cause a change in settings of the respiratory therapy system 120 based at least in part on the result associated with the first and the second scores. For example, the result can indicate that the second score is objectively worse than the first score, so for a next sleep session, the respiratory therapy system 120 can cause an adjustment to (i) a pressure setting of supplied pressurized air provided by the respiratory therapy device 122, (ii) a humidity setting of the supplied pressurized air provided by the respiratory therapy device 122, or (iii) both (i) and (ii).

In some implementations, the steps in method 500 are repeated over a period of time to obtain a trend of scores for a total number of tests performed. The control system 110 can cause an adjustment to the pressure setting or the humidity setting of the supplied pressurized air based at least in part on the trend of scores indicating that the user 210 is performing objectively better or worse over the period of time.

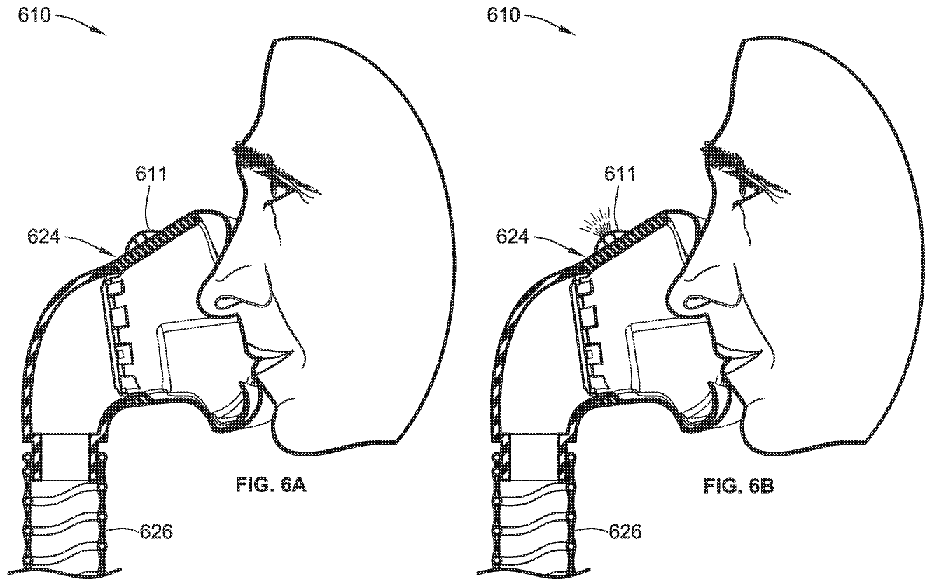

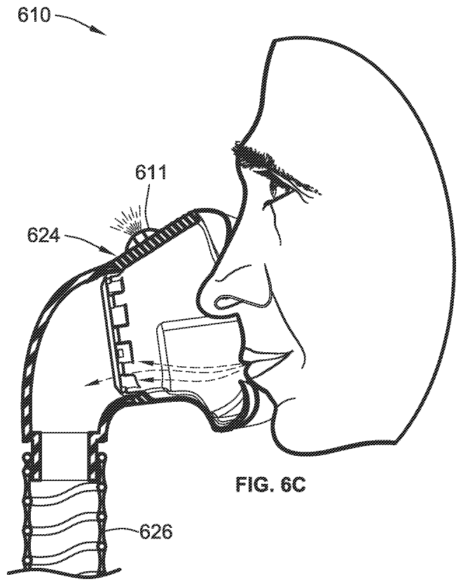

Referring to FIGS. 6A-6C, a sequence for performing a reactive time test is illustrated. FIG. 6A illustrates a user 610 wearing a user interface 624, according to some implementations of the present disclosure. The user interface 624 is the same as, or similar to, the user interface 124 described herein. The user interface 624 can include a cushion, a frame, a connector (to connect the frame to a conduit), a plurality of straps, or any combination thereof. FIG. 6A illustrates a cross-sectional cut out of the user interface 624. The user interface 624 is connected to a conduit 626 (which is the same as, or similar to, the conduit 126) and is connected to a respiratory therapy device (e.g., the respiratory therapy device 122 in FIG. 1). The user interface 624 includes a light source 611 (e.g., one or more LED lights) that is able to emit one or more colors of light.

In FIG. 6B, the user 610 receives a light stimulus via the light source 611, according to some implementations of the present disclosure. The light source 611 turning on is a visual indicator (i.e., the light stimulus) for the user 610 to provide a response. In FIGS. 6A and 6B, the user 610 is in a normal breathing pattern or a normal breathing cycle prior to the visual indicator being provided. That is, the user 610 can be breathing in or out depending on which point the user 610 is at in her breathing cycle. The breathing of the user can be through her nose and/or mouth in FIGS. 6A and 6B.

FIG. 6C illustrates the user 610 purposefully responding to the light stimulus, according to some implementations of the present disclosure. In FIG. 6C, the user 610 blows into the user interface 624 as indicated by the arrows from the lips/mouth of the user 610. The arrows indicate that the user 610 expels (e.g., blows, puffs, etc.) air current at a higher flow rate than in the normal breathing pattern of the user 610. In some implementations, the flow rate sensor 134 and/or the pressure sensor 132 integrated in the respiratory therapy system 120 can be used to determine that the user 610 has expelled air current. In some implementations, a sensor external to the respiratory therapy system 120 is used to determine that the user 610 has expelled air current. The sensor may be communicatively coupled to the respiratory therapy system 120 and/or the respiratory therapy device 122.

The method illustrated in FIGS. 6A to 6C is one exemplary implementation of the concepts of the present disclosure where the light source 611 provides a stimulus and the expelled air by the user 610 provides a response to that stimulus. As described herein, the time between the stimulus and the response can be used to measure a current psychomotor reaction time (or the like) of the user 610.

In some implementations, a reaction time test for a specific individual need not be captured. A machine learning model can be trained with data from one or more individuals and can be used to estimate how the specific individual would have performed on a reaction time test. Some advantages to using a trained machine learning model for reaction time estimates or predictions include (a) relieving specific individuals from being required to take reaction time tests over extended periods of time, (b) relaxing data requirements for estimating reaction time tests since data from a plurality of individuals can be leveraged, (c) ability to estimate reaction times from passive data, that is, data that do not involve a specific individual providing an input, and (d) ability to forecast predicted reaction times forward in time. The present disclosure provides reliable ways of estimating reaction times and using these estimates to suggest therapy changes to an individual.

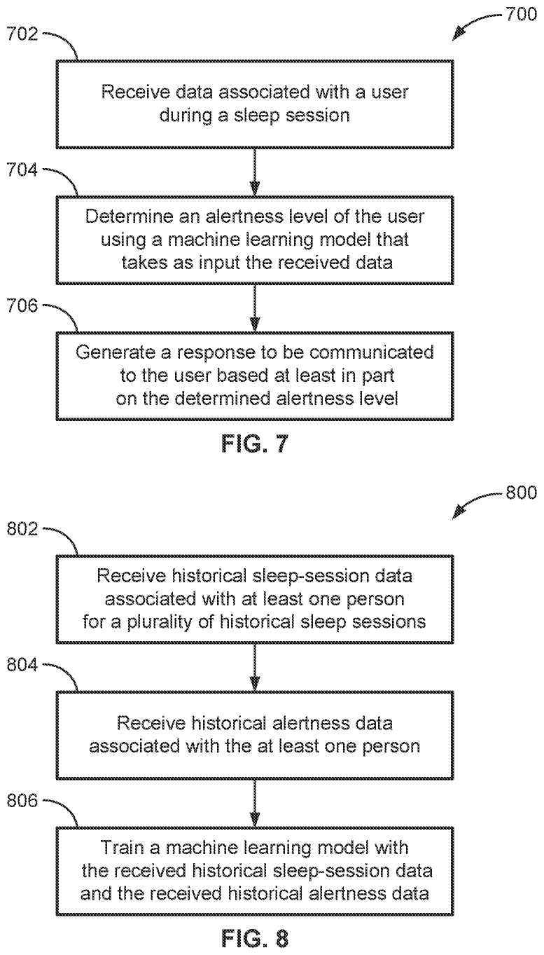

FIG. 7 is a flow diagram for generating a response for a user based on an alertness level of the user, according to some implementations of the present disclosure. The method 700 can be implemented using any combination of elements or aspects of the system 100 described herein.

Step 702 involves receiving data associated with a user (e.g., the user 210) during a sleep session. In some implementations, the control system 110 receives the data from the respiratory therapy system 120, the sensor 130, the user device 170, or any combination thereof. The data received can include a flow signal, a respiration signal, a respiration rate, a respiration rate variability, an inspiration amplitude, an expiration amplitude, an inspiration-expiration ratio, or any combination thereof. The flow signal from the flow rate sensor 134 can be used to derive a respiratory flow signal that indicates volumetric flow rate of air inhaled and exhaled by the user 210. The respiration rate is a number of breaths the user 210 takes per unit time (e.g., 15 breaths per minute, 20 breaths per minute, etc.) where a breath consists of an inhalation followed by an exhalation. The inspiration amplitude and the expiration amplitude can be volumetric measures of air during inspiration and expiration cycles while the user 210 is breathing.

The flow rate sensor 134, the pressure sensor 132, the temperature sensor 136, or any combination thereof, can be used to measure some of these respiration measures. The control system 110 can derive some of these respiration measures (e.g., the inspiration-expiration ratio can be derived by dividing the inspiration amplitude by the expiration amplitude). In some embodiments, the inspiration-expiration ratio can be determined as a ratio of time consumed by inspiration and a ratio of time consumed by expiration.

At step 702, the received data can include flow and/or pressure settings of the respiratory therapy system 120, a heart rate of the user 210, heart rate variability of the user 210, blood pressure of the user 210, blood pressure variability of the user 210, movement of the user 210, or any combination thereof. Measurements of these parameters using the system 100 are already described in connection with FIG. 1.

The received data at step 702 can include a sleep stage, a sleep state, a duration the user spends in the sleep state, a duration the user spends in the sleep stage, a ratio of the duration the user spends in the sleep state to a duration of the sleep session, a ratio of the duration the user spends in the sleep stage to the duration of the sleep session, a ratio of a first sleep state to a second sleep state, a ratio of a first sleep stage to a second sleep stage, or any combination thereof. As discussed in connection with FIG. 1, the control system 110 can determine sleep states and/or sleep stages of the user 210 using physiological data generated by the sensors 130. Once sleep states and/or sleep stages are determined for the sleep session, the other parameters such as ratios of duration of one sleep state (and/or stage) to another or duration of each sleep state (and/or stage) can be determined. Example ratios include a light sleep ratio, a deep sleep ratio, a REM sleep ratio, etc.

The received data at step 702 can include a number of events per hour, a pattern of the events, a duration of each of the events, or any combination thereof. Examples of events include central apneas, obstructive apneas, mixed apneas, hypopneas, snoring (such as simple or complex), periodic limb movement, awakenings, chokings, epileptic episodes, seizures, or any combination thereof. The flow rate sensor 134 can be used to measure snoring oscillation. The received data can include apnea-hypopnea index (AHI) which is a measure sleep apnea severity determined as a number of apneas and hypopneas that occur on average per unit time (e.g., per hour, per day, etc.). The received data can include residual AHI, a ratio of on-therapy residual AHI to off-therapy AHI, or both. Residual AHI can be defined as remaining events that are detected (and not effectively treated) by the respiratory therapy device 122 operating in continuous pressure mode, or in an auto adjusting mode. Preferably, this residual AHI is kept as low as possible, e.g., under an AHI of 5. A residual AHI above zero (and say above 5 or 10) can discourage users from continuing therapy since the users may not perceive the benefits of therapy, and may have reduced alertness compared to a desired alertness level. Even a low residual AHI can indicate potential alertness issues, such as group or train of apneas occurring in REM sleep shortly before wakeup time, which could give rise to a feeling of unease, grogginess, bad temperedness due to the stress/sympathetic activation caused by the untreated apnea/hypopneas. Equally, a bout of high mask leak or mouth leak close to wake up time can have a negative impact on short term alertness and feeling of wellness (or lack thereof)—akin to the person feeling that they “have got out of bed on the wrong side”—i.e., irritable, with no easy explanation of why. Sympathetic activation as exemplified via galvanic skin response (GSR) or heart rate variability (HRV) metrics, along with residual apnea detection on the respiratory therapy device 122 can help uncover these links of perceived angst and estimated short term reduced alertness levels. In contrast, a good balance of restorative sleep, with a reasonable proportion and duration of REM and deep sleep can give rise to a feeling of being refreshed, ready for the day, a predominant vagal or parasympathetic activation on initial wakeup.

For a sleep session, “on-therapy” describes the user 210 being asleep and engaged to the respiratory therapy system 120 during the sleep session, and “off-therapy” describes the user 210 being asleep and not engaged to the respiratory therapy system 120 during the sleep session. For example, during the sleep session, the respiratory therapy system 120 can generate sleep related data for the user 210, and external sensors in the sensors 130 can also generate sleep related data for the user 210. When the control system 110 receives sleep related data for the user 210 from only external sensors and not from the respiratory therapy system 120, the control system 110 can determine that the user 210 is off-therapy. A period of time where the sleep related data from the respiratory therapy system 120 is received can be determined by time stamping when the respiratory therapy system 120 starts providing sleep related data and when the respiratory therapy system 120 stops providing the sleep related data.

The received data at step 702 can include therapy efficacy, a sleep efficiency, a bedtime of the user 210, a ratio of on-therapy sleep efficiency to off-therapy sleep efficiency, a ratio of on-therapy sleep duration to a duration of the sleep session, a ratio of on-therapy sleep duration to off-therapy sleep duration, or any combination thereof. Therapy efficacy includes whether an unintentional leak associated with the respiratory therapy system 120 during the sleep session is below a threshold. Sleep efficiency provides a metric of how well the user 210 has slept during the sleep session and can be measured as a ratio of the duration of time the user 210 spends asleep to a duration the user 210 is in bed. For example, if the user 210 is asleep for 6 hours but spends 8 hours in bed, sleep efficiency can be provided as 75%.

The received data at step 702 can include a sleep score, a mind recharge score, a body recharge score, or any combination thereof. Examples of such scores, and how to calculate such scores, is described in, for example, WO 2015/006364, which is hereby incorporated by reference herein in its entirety. The sleep score can be calculated using one or more sleep parameters already described herein. In some implementations, the sleep score involves a weighting of different parameters. The mind recharge score can be based on a proportion of REM sleep for the user 210 for the sleep session related to a proportion of REM sleep for average users in a same demographic as the user 210. The body recharge score can be based on a proportion of deep sleep for the user 210 for the sleep session related to a proportion of the deep sleep for average users in a same demographic as the user 210. Demographic information includes age, sex, geographic location, etc.

In some implementations, the control system 110 determines the sleep score based on measured data representing movement of the user 210, total sleep time, deep sleep time, REM sleep time and light sleep time, wake after sleep onset time and sleep onset time. In some cases, the features may include time domain statistics and/or frequency domain statistics. The sleep score may include a total having a plurality of component values, each component value determined with a function of a measured sleep factor and a predetermined normative value for the sleep factor. The function may include a weighting variable varying between 0 and 1 and wherein the weighting is multiplied by the predetermined normative value. The function of at least one sleep factor for determining a component value may be an increasing function of the measured sleep factor, such as when the at least one sleep factor is one of total sleep time, deep sleep time, REM sleep time and light sleep time. In some cases, the function of at least one sleep factor for determining a component value may be an initially increasing and subsequently decreasing function of the measured sleep factor, such as when the at least one sleep factor is REM sleep time. The function of at least one sleep factor for determining a component value may be a decreasing function of the measured sleep factor, such as, when the at least one sleep factor is one of sleep onset time and wake after sleep onset time.

In some implementations, the control system 110 communicates with a clinician/health care professional (HCP) if a reduced alertness or vigilance is detected or estimated for the day. In an example, the user may be in a safety critical role (e.g., a heavy machinery or public transport operator) and can pose a risk or hazard due to their reduced alertness. This could include the HCP recommending or setting changes on the respiratory therapy device 122 (such as adapting or changing a program), changing medication, and/or recommending that the user refrain from certain high risk tasks pending further on site assessment.

In some implementations, the control system 110 obtains other information from the user 210 that can affect sleep. For example, the control system 110 can cause a prompt to be provided to the user 210 such that the user 210 inputs one or more of daily caffeine consumption, daily alcohol consumption, daily stress level, and daily exercise amount. A sleep score can capture aspects of one or more of: an effectiveness of the user interface 124 (e.g., low leak and/or good seal), usage time of respiratory therapy device 122, reduced awakenings or arousals, ratio of deep sleep, ratio of REM sleep, apneas effectively treated without disturbing the user 210, residual AHI, subject inputs obtained from the user 210 (e.g., based on how the user 210 feels), sufficiency of sleep cycles, snoring, residual snoring after a therapy, sleep efficiency, sleep quality, sleep latency, sleep fragmentation, comparison to people of similar age, comparison to people of similar gender, etc.

Step 704 involves determining an alertness level of the user 210 using a machine learning model that takes as input the received data from step 702. The determined alertness level can be an alertness level right when the user 210 wakes from sleep. The determined alertness level can be an alertness level at a future time after the user 210 wakes from sleep. For example, the user 210 wakes up at 7 AM, and the determined alertness level is provided for 3 PM. Alertness level determinations for the future time of day can be windowed (e.g. 2 hours ahead, 12 hours ahead, 16 hours ahead, 24 hours ahead, etc.). Alertness level determinations in the future can be obtained approximately the same time after the user 210 wakes from sleep. In some implementations, the determined alertness level includes a trend that provides multiple alertness levels. For example, the user 210 wakes up at 7 AM, and the determined alertness level includes multiple alertness levels for 10 AM, 2 PM, 5 PM, 7 PM, and 9 PM. In some implementations, the control system 110 determines the alertness level of the user 210 as an alertness score, as further described herein. Such a score may be based on, for example, an assessment of reaction time(s) measured following a stimulus and compared to expected reaction time(s), which expected reaction time(s) may be derived from an individual's historical reaction time data, population-level reaction time data (optionally matched based on the individual's demographic and/or medical parameter), or a combination thereof. The alertness score may also be related to a circadian rhythm-based model of sleepiness. Other factors such as diagnosed insomnia, OSA, CSA, delayed sleep phase syndrome (e.g., “social jetlag”), insufficient sleep syndrome (e.g., excessive sleep debt), and/or depression can be utilized in calculating the alertness score.

Step 706 involves generating a response to be communicated to the user 210, optionally via a third party such as a doctor, based at least in part on the determined alertness level. In some implementations, the generated response is a message including the determined alertness level, and the control system 110 causes the message to be provided to the user 210 via the speaker 142, the display device 128, the display device 172, or any combination thereof. In some implementations, the generated response is an alarm such as a visual alarm provided by a light source coupled to the respiratory therapy system 120, an auditory alarm provided by the speaker 142, and/or a vibratory alarm provided by vibrating the user device 170 and/or the respiratory therapy device 122.

At step 706, in some implementations, a predicted length of the remaining duration of the sleep session is taken into account on whether to generate the response. For example, if the user 210 is expected to wake up in twenty minutes, then the control system 110 does not generate the response. On the other hand, if the user 210 is expected to wake up in forty-five minutes or more, then the control system 110 generates the response. For example, if the user 210 wakes up for a bathroom break, the response can be an alarm or a message instructing the user 210 to engage with the respiratory therapy system 120 prior to going back to sleep. The control system 110 can thus take advantage of a regular waking time or a regular bed time of the user 210 to determine the remaining duration of the sleep session. That is, the typical sleep duration of the user 210 can be used to predict the remaining duration of the sleep session.

In some implementations, the control system 110 generates the response based on the user interface 124 not being securely engaged to the user 210 and the remaining duration of the sleep session is greater than a sleep duration threshold. For example, the user 210 may engage with the user interface 124 in a manner that an excess of the supplied pressurized air leaks from the user interface 124. Excessive leakage is air leakage from the user interface 124 in excess of expired air including carbon dioxide (CO₂) from the breathing of the user 210. The microphone 140 and/or the pressure sensor 132 can be used to determine excessive leakage in the user interface 124. When the user 210 sleeps with the user interface 124 not securely engaged, the control system 110 can estimate the remaining duration of the current sleep session. If the remaining duration is above the sleep duration threshold, then the control system 110 generates the response. The sleep duration threshold can be forty-five minutes, an hour, etc. The response can be an alarm to wake the user 210 to properly engage with the respiratory therapy system 120. The response can include a message instructing the user 210 to properly engage with the respiratory therapy system 120.

In some implementations, the sleep duration threshold is determined based on a likelihood that the determined alertness level can be improved by therapy during the remaining duration of the sleep session. The likelihood that the determined alertness level can be improved can be calculated from, for example, historical data as a number of apnea events suffered by the user 210 per unit time when the user interface 124 is not securely engaged to the user 210 multiplied by the remaining duration of the current sleep session. The likelihood of improvement can also be determined using the machine learning model by probing the machine learning model with various on-therapy durations for the remaining duration of the sleep session. The determined likelihood can be compared against a threshold to determine whether to generate the response. For example, if the determined likelihood is greater than 0.90, then the control system 110 generates the response. The response can further be generated based on the magnitude of a potential improvement on the determined alertness level being above an improvement threshold. That is, if the determined alertness level is an alertness score of 80/100 and the magnitude of improvement is determined to be a 10 such that the user 210 can potentially reach 90/100, then the control system 110 can determine that 10 is greater than 5 (which is the improvement threshold in this example) and generate the response.

In some implementations, at step 706, the generated response is further based at least in part on a duration of the sleep session. The control system 110 can determine a likelihood that the determined alertness level can be improved by extending the duration of the sleep session. The likelihood can be calculated based on the machine learning model by probing the machine learning model with potentially longer sleep durations. The control system 110 can generate the response based on the likelihood being above a threshold, a magnitude of potential improvement on the determined alertness level being above an improvement threshold, or both. The generated response can include a message to the user instructing the user to go back to bed for ten minutes, thirty minutes, an hour, two hours, etc., to extend the sleep session. The generated response can include a message to the user 210 instructing the user to sleep for ten minutes longer, twenty minutes longer, an hour longer, etc., during a future sleep session. For example, if the user 210 had five hours of sleep, then the control system 110 can instruct the user 210 to sleep for seven hours next time.

In some implementations, at step 706, the generated response is further based at least in part on a duration of a portion of the sleep session that the user is not engaged with the user interface 124. That is, after the user 210 wakes from sleep, the control system 110 determines a duration when the user 210 was on-therapy and a duration when the user 210 was off-therapy for the sleep session. The control system 110 determines whether to generate the response based on the duration where the user 210 was on-therapy or off-therapy. In some implementations, the control system 110 determines a likelihood that the determined alertness level can be improved during a future sleep session by increasing the duration of on-therapy sleep or reducing the duration of off-therapy sleep. In some implementations, the likelihood can be determined by, for a same total number of hours of sleep, using different durations of off-therapy and on-therapy sleep with the machine learning model to determine whether the determined alertness level is improved. In some implementations, the total number of hours of sleep is increased or decreased when determining whether the determined alertness level is improved. The control system 110 can generate the response based on the likelihood being above a threshold, a magnitude of potential improvement on the determined alertness level being above an improvement threshold, or both. The generated response can be a message instructing the user 210 to don the user interface 124 prior to a future sleep session. The generated response can be a message instructing the user 210 to reduce a duration of the future sleep session when the user interface 124 is donned. For example, the control system 110 determines that the user 210 slept for eight hours, but for five of those eight hours, the user 210 was not engaged to the user interface 124. The control system 110 can then recommend that for the next sleep session that the user 210 sleep for six and half hours, seven hours, seven and a half hours, etc., but don the user interface 124 for the entire duration. In some embodiments, the user 210 suffers from severe AHI and may be in bed for twelve to fourteen hours with a portion of the time in bed not engaged to the user interface 124. The control system 110 can recommend that the user 210 engage with the user interface 124 to reduce the amount of time in bed to a healthier seven to nine hours of sleep. As such, reducing off-therapy time can result in reducing the total duration of the future sleep session required to achieve a similar or better alertness level. The amount of reduction recommended by the control system 110 can be based at least in part on an age of the user 210.

In some implementations, at step 706, the control system 110 generates the response based at least in part on the determined alertness level of step 704 satisfying a condition. The condition can include the determined alertness level being (i) below an average alertness level for the user, (ii) below a threshold, or (iii) above the threshold. For example, the determined alertness level can be an alertness score of 75 which is compared against a threshold of 80, and the response is generated because the alertness score is less than the threshold 80. In some implementations, the condition can include the determined alertness level at a future time of day being (i) below an average alertness level for the user, (ii) below a threshold, or (iii) above the threshold. For example, the determined alertness level can be an alertness score of 75 at 3 PM which is compared against a threshold of 70, and the response is generated because the alertness score at 3 PM is greater than the threshold 70. The response can be a message to the user 210 informing the user 210 that at the determined alertness level (for the future point in time or for a current time) exceeds the threshold and that the user 210 had a good night sleep. The response can be a message to the user 210 informing the user 210 that the determined alertness level (for the future point in time or for the current time) is below the threshold and that the user 210 should use more therapy or should implement a bedtime routine to get a better night sleep such that a future alertness level can be improved.

In some implementations, the generated response is an encouraging message. An example message is “Your current alertness score is 50. Remember to use your respiratory therapy device to get your alertness score to 70 when you wake up at 6 AM.” In another example, the message is “Yesterday, your alertness score at 2 PM was 55, use your respiratory therapy device for at least 5 hours to improve your alertness score tomorrow afternoon.” In another example, the message is, “You can improve your alertness level by sleeping an hour more.”

In some implementations, the generated response is further based in part on therapy efficacy during the sleep session, therapy efficacy during past sleep sessions, or both. Therapy efficacy can be related to excessive leakage as previously discussed. Hence, the response generated can be a message instructing the user to improve therapy efficacy for a future sleep session or for the current sleep session by: (i) increasing an upper pressure limit of the supplied pressurized air provided by the respiratory therapy device 122, or decrease the upper pressure limit of the supplied pressurized air; (ii) increase a lower pressure limit of the supplied pressurized air, or decrease the lower pressure limit of the supplied pressurized air; (iii) replace the user interface 124 coupled to the respiratory therapy device 122; (iv) adjust a humidity of the supplied pressurized air; or (v) any combination of (i)-(iv).

Various implementations of the present disclosure as described in connection with FIG. 7 relied on the machine learning model for determining alertness level. Referring to FIG. 8, a flow diagram for training the machine learning model to predict the alertness level of a target user (e.g., the user 210) is provided, according to some implementations of the present disclosure. The method 800 can be implemented using any combination of elements or aspects of the system 100 described herein.

Step 802 involves receiving historical sleep-session data associated with at least one person for a plurality of historical sleep sessions. The at least one person can include (i) one or more people associated with or using a respiratory therapy system, (ii) one or more people that do not use a respiratory therapy system, or (iii) both (i) and (ii). For a respective sleep session in the plurality of historical sleep sessions, historical sleep-session data associated with each of the at least one person can include a sleep score, a mind recharge score, a body recharge score, a flow signal, a respiration signal, a respiration rate, a respiration rate variability, an inspiration amplitude, an expiration amplitude, an inspiration-expiration ratio, a number of events per hour, a pattern of the events, a duration of each of the events, a sleep state and/or sleep stage, a duration the person spends in the sleep state and/or sleep stage, a ratio of the duration the person spends in the sleep state and/or sleep stage to a duration of the respective sleep session, a ratio of a first sleep state to a second sleep state, a ratio of a first sleep stage to a second sleep stage, a bedtime of the person, residual AHI, pressure settings of a respiratory therapy system, a heart rate, a heart rate variability, a blood pressure, a blood pressure variability, movement of the person, sleep efficiency, therapy efficacy, a ratio of on-therapy sleep duration to off-therapy sleep duration, a ratio of on-therapy sleep duration to the duration of the respective sleep session, a ratio of on-therapy residual AHI to off-therapy AHI, a ratio of on-therapy sleep efficiency to off-therapy sleep efficiency, or any combination thereof. Each of these parameters was previously described in connection with step 702 of FIG. 7. The parameters can be received from sensors, user devices, respiratory therapy devices and/or respiratory therapy systems associated with the at least one person. In some implementations, the historical sleep-session data includes subjective feedback from the at least one person. The subjective feedback can be one or more ratings for how the at least one person grades one or more historical sleep sessions in the plurality of historical sleep sessions.

In some implementations, the at least one person is one person, two people, five people, one hundred people, one thousand people, ten thousand people, a million people, a billion people, etc. In some implementations, the at least one person includes the user 210 such that, the historical sleep-session data includes historical sleep-session data of the user 210. In some implementations, the at least one person consists of only one person which is the user 210. In some implementations, the at least one person does not include the user 210.

In some implementations, the at least one person are people in a cohort. The cohort can be based at least in part on health condition(s) shared by the at least one person. For example, individuals with diabetes can be grouped in a cohort, individuals with high blood pressure can be grouped in a cohort, individuals with insomnia can be grouped in a cohort, etc. The cohort can be based on demographic information. For example, individuals falling between age 18 and 25 can be grouped in a cohort, individuals between age 40 to 50 can be grouped in a cohort, individuals of a same ethnic group or having certain genetic markers can be grouped in a cohort, individuals in a same geographical location can be grouped in a cohort since they may be influenced by similar environmental factors, etc. The target user (e.g., the user 210) can share the same health condition(s) and/or demographic information as the cohort.

Step 804 involves receiving historical alertness data associated with the at least one person. The alertness data includes alertness levels associated with each of the at least one person outside of the plurality of historical sleep sessions. Being outside of a sleep session includes immediately after the sleep session, or hour(s), day(s), week(s), etc., after the sleep session. The alertness data can include results from sustained attention, reaction time tests (e.g., a psychomotor vigilance task test) as described in above in connection with some implementations of the present disclosure. The results from the reaction time tests can be measured in milliseconds and can be timestamped for different times of day. The results from the reaction time tests can be pruned or cleaned to remove missed responses to stimuli, to remove false starts, etc. The results from the reaction time tests can be quoted as average response times, median response times, etc.

The alertness data can have an associated timestamp to indicate a time of day. For example, the alertness data can include reaction time tests performed by the at least one person during morning hours, during afternoon hours, during evening hours, or any combination thereof. In some implementations, the alertness data is accompanied by one or more features associated with the at least one user. For example, a respective person in the at least one user can perform a reaction time test at 3 PM, and the result of the reaction time test along with physiological data associated with the respective person is included in the alertness data. Physiological data can include average heartrate value, change in heartbeat, spectral analysis of expired air, etc. Physiological data can be obtained from the one or more sensors 130 as discussed in connection with some embodiments of the present disclosure.

In some implementations, the physiological data included in the alertness data can be windowed based on the type of physiological data. For example, 60 heartbeats can be obtained in a minute of data collection while about 15 breaths can be obtained in a minute of data collection. As such, to obtain a representative sample of the physiological data around when the respective person attempted the reaction time test, the physiological data can be obtained in a 30-second window, a 5-minute window, etc. In some implementations, physiological data for a most recent sleep session prior to the reaction time test is linked and tagged with the results of the physiological data to be included in the alertness data.

Step 806 involves training the machine learning model with the received historical sleep-session data of step 802 and the received historical alertness data of step 804. Once trained, the machine learning model is configured to (i) receive as an input current sleep-session data associated with the user 210 and (ii) determine as an output a predicted alertness level associated with the user 210 at one or more points in time.

In some implementations, at step 806, the control system 110 correlates the received historical sleep-session data and the received historical alertness data to determine features or qualities of the parameters within the historical sleep-session data that indicate good or bad reaction times, indicative of alertness levels. The control system 110 can determine thresholds for good reaction times and bad reaction times for the at least one person.

In some implementations, at step 806, the control system 110 correlates on-therapy data for the at least one person present in the historical sleep-session data with physiological data present in the historical alertness data to determine how respiratory therapy device usage during the plurality of historical sleep sessions affects physiological data outside of the plurality of historical sleep sessions. The control system 110 can align periods of bad reaction times in the historical alertness data with the historical sleep-session data such that changes in sleep (e.g., sleep efficiency, on-therapy to off-therapy ratios, etc.) can be shown to affect the alertness data. One or more techniques can be used for the analysis. For example, decision trees, bootstrap aggregation, log-linear fit, support vector machines, or any combination thereof, can be used to align the sleep data and develop the relationship between the sleep data and the alertness data.

In some implementations, the machine learning model is trained in successive iterations such that historical sleep-session data associated with a first one of the at least one person and historical alertness data associated with the first one of the at least one person are used to train the machine learning model in a first iteration. After the first iteration, the machine learning model is tuned specifically to the first one of the at least one person. For successive iterations, historical sleep-session data associated with subsequent persons and historical alertness data associated with the subsequent persons are used to update the machine learning model in subsequent iterations. After a respective iteration, the machine learning model reduces an error associated with a respective one of the at least one person when predicting an alertness level associated with the respective one of the at least one person.

Each successive training iteration is an update to the machine learning model. For example, the control system 110 can store in memory a trained machine learning model for predicting alertness levels. The machine learning model was trained using historical sleep-session data and historical alertness data from multiple individuals not including the user 210. The user 210 can then provide historical sleep-session data associated with the user 210 for at least one previous sleep session to the control system 110. The user 210 can also provide historical alertness data associated with the user 210 outside of the at least one previous sleep session. The control system 110 can then update the machine learning model by further training the trained machine learning model with the historical sleep-session data and the historical alertness data associated with the user 210. Prior to updating the trained machine learning model, if the user 210 used the trained machine learning model to obtain an alertness level, the trained machine learning model had an error level (or an error rate) of e1 associated with the user 210. After updating the trained machine learning model, the trained machine learning model has an error level of e2 associated with the user 210, where e2 is less than e1.

In some implementations, the alertness level predicted by the trained machine learning algorithm is provided as an alertness score. The alertness score takes into account not just raw alertness data but also health condition and/or demographic information. For example, the alertness score can be quoted as 70 for both a 70-year-old and for a 25-year-old even though the 25-year-old had much faster reaction times compared to the 70-year-old. The quoted 70 alertness score takes into account the age of the individuals, and as such, the alertness score embeds a comparison between expected performance of those in a same cohort as the 25-year-old and specific performance of the 25-year-old. Similarly, health condition can be incorporated to adjust the alertness score.

The following example steps can be used in training the machine learning model. The historical sleep-session data and the alertness data used for training can be conditioned by rejecting outliers in the data. Short sleep data in the historical sleep-session data can be removed. Short sleep data includes sleep duration less than 10 minutes, 15 minutes, 30 minutes, 1 hour, etc. After conditioning a regression algorithm can be applied to the conditioned data. In some implementations, ensemble learning with bagging using decision trees is the technique used for learning. A number of parameters or variables from the historical sleep-session data are selected at random for each decision split in the decision tree. Empirical feature importance is estimated for features that contributed to specific reaction time results in the historical alertness data. In some implementations, six to eight features are identified for each of the specific reaction time test results. Empirical feature importance can help reduce dimensionality of the historical sleep-session data being considered. In some implementations, AHI and respiration rate are important or controlling features for predicting alertness using sleep data.

One or more elements or aspects or steps, or any portion(s) thereof, from one or more of any of claims 1-168 below can be combined with one or more elements or aspects or steps, or any portion(s) thereof, from one or more of any of the other claims 1-168 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving data associated with an individual during a sleep session;
determining an alertness level of the individual using a machine learning model that takes as input the received data; and
generating a response to be communicated to the individual based at least in part on the determined alertness level.

2. The method of claim 1, wherein the received data includes a sleep score, a mind recharge score, a body recharge score, a flow signal, a respiration signal, a respiration rate, a respiration rate variability, an inspiration amplitude, an expiration amplitude, an inspiration-expiration ratio, a number of events, a number of the events per unit time, a pattern of the events, a duration of each of the events, a sleep stage, a duration the individual spends in the sleep stage, a ratio of the duration the individual spends in the sleep stage to a duration of the sleep session, a ratio of a first sleep stage to a second sleep stage, a bedtime of the individual, residual apnea-hypopnea index (AHI), pressure of supplied pressurized air during therapy, flow rate of the supplied pressurized air during therapy, a heart rate, a heart rate variability, a blood pressure, a blood pressure variability, movement of the individual, sleep efficiency, therapy efficacy, a ratio of on-therapy sleep duration to off-therapy sleep duration, a ratio of on-therapy sleep duration to the duration of the sleep session, a ratio of on-therapy residual AHI to off-therapy AHI, a ratio of on-therapy sleep efficiency to off-therapy sleep efficiency, or any combination thereof.

3-4. (canceled)

5. The method of claim 2, wherein the therapy efficacy is based at least in part on an unintentional leak of the supplied pressurized air during the sleep session being below a threshold.

6. The method of claim 1, wherein the generated response is further based at least in part on a remaining duration of the sleep session.

7. The method of claim 6, wherein the generated response is further based at least in part on the individual not being engaged with a user interface of a respiratory therapy system and the predicted remaining duration of the sleep session being above or below a sleep duration threshold.

8. (canceled)

9. The method of claim 7, wherein the generated response includes (a) an alarm to wake the individual to don the user interface or (b) a message instructing the individual to don the user interface prior to going back to sleep.

10-11. (canceled)

12. The method of claim 1, further comprising:
determining a likelihood that the determined alertness level can be improved by extending a duration of the sleep session;
wherein the generated response is further based at least in part on (i) the likelihood being above a threshold, (ii) a magnitude of a potential improvement on the determined alertness level being above an improvement threshold, (iii) the duration of the sleep session, or (iv) any combination of (i)-(iii).

13. The method of claim 11, wherein the generated response is a message instructing the individual to (a) extend the duration of the sleep session, (b) extend a duration of a future sleep session, or (c) both (a) and (b).

14. (canceled)

15. The method of claim 1, wherein the generated response is further based at least in part on a duration of a portion of the sleep session that the individual is not engaged with a user interface of a respiratory therapy system.

16-17. (canceled)

18. The method of claim 15, wherein the message further instructs the individual to reduce a duration of the future sleep session during which the user interface is donned.

19-22. (canceled)

23. The method of claim 1, wherein the determined alertness level is a predicted alertness level for a future time of day following the sleep session.

24. (canceled)

25. The method of claim 1, wherein the data associated with the individual are received from (i) a respiratory therapy device configured to supply pressurized air to an airway of the individual by way of a user interface coupled to the respiratory therapy device via a conduit, (ii) a sensor, or (iii) both (i) and (ii).

26. The method of claim 25, further comprising:
determining that the individual was off therapy during at least a portion of the sleep session, based at least in part on receiving first data from the sensor and not receiving second data from the respiratory therapy device at a same time during the portion of the sleep session,
wherein the data associated with the individual comprise the first data and the second data.

27-114. (canceled)

115. A system comprising:
a respiratory therapy device configured to supply pressurized air to an airway of a user, the pressurized air being supplied by way of a user interface coupled to the respiratory therapy device via a conduit;
a memory storing machine-readable instructions; and
a control system including one or more processors configured to execute the machine-readable instructions to:
cause a test to begin, the test including causing a stimulus to be generated at a first point in time;
receive a response to the stimulus from the user at a second point in time, the response including an expelled air current from the user that is detected using the respiratory therapy device; and
determine a first score based at least in part on (i) the first point in time and (ii) the second point in time.

116. (canceled)

117. The system of claim 115, wherein the test is a reaction time test, and the reaction time test is a sustained attention, reaction time test.

118. (canceled)

119. The system of claim 117, wherein the sustained attention, reaction time test includes a plurality of stimuli and is implemented over a period of time.

120-121. (canceled)

122. The system of claim 115, wherein the expelled air current is detected using (i) a flow sensor coupled to the respiratory therapy device, (ii) a microphone coupled to the respiratory therapy device, (iii) a pressure sensor coupled to the respiratory therapy device, or (iv) any combination of (i), (ii), and (iii).

123. (canceled)

124. The system of claim 115, wherein the stimulus generated at the first point in time is generated by (i) a light source coupled to the respiratory therapy device, the conduit, the user interface, or any combination thereof, (ii) a speaker coupled to a housing of the respiratory therapy device, the conduit, or the user interface, or (iii) both (i) and (ii), and wherein the control system is further configured to execute the machine-readable instructions to:
receive a second score from an electronic device associated with the user, the second score being determined from a second test performed by the electronic device.

125. The system of claim 124, wherein the control system is further configured to execute the machine-readable instructions to determine a normalized relationship for relating the second score to the first score.

126-128. (canceled)

129. The system of claim 115, wherein the stimulus includes light generated by a light source, (b) sound generated by a speaker, (c) vibration generated by a motor of the respiratory therapy device, or (d) any combination of (a)-(c). 130-136. (canceled)

137. The system of claim 115, wherein the stimulus includes varying a pressure of the pressurized air, supplied by the respiratory therapy device, from a first pressure to a second pressure.

138. The system of claim 137, wherein the stimulus further includes varying the pressure of the pressurized air back to the first pressure.

139. The system of claim 115, wherein the stimulus includes the respiratory therapy device (a) stopping supply of the pressurized air to the user or (b) starting the supply of the pressurized air to the user.

140. (canceled)

141. The system of claim 115, wherein the control system is further configured to execute the machine-readable instructions to determine the first score based at least in part on an elapsed time between the first point in time and the second point in time.

142. The system of claim 141, wherein the control system is further configured to execute the machine-readable instructions to:
determine a correction factor for adjusting the elapsed time, the correction factor based at least in part on a time delay in generating the stimulus, the time delay including (i) a delay in a light stimulus turning on and being visible, (ii) a delay in a sound stimulus being generated, or (iii) both;
adjust the elapsed time using the correction factor; and
determine the first score based at least in part on the adjusted elapsed time.

143. The system of claim 141, wherein the control system is further configured to execute the machine-readable instructions to:
determine a correction factor for adjusting the elapsed time, the correction factor based at least in part on a time delay in the user responding to the stimulus due to the stimulus being generated at a point in a breathing cycle of the user;
adjust the elapsed time using the correction factor; and
determine the first score based at least in part on the adjusted elapsed time.

144-151. (canceled)

152. A system comprising:
a respiratory therapy system including a respiratory therapy device, a conduit, and a user interface, the respiratory therapy device being configured to supply pressurized air to an airway of a user by way of the user interface that is coupled to the respiratory therapy device via the conduit;
a memory storing machine-readable instructions; and
a control system including one or more processors configured to execute the machine-readable instructions to:
cause a first test to begin, the first test including causing a first stimulus to be generated at a first point in time;
receive a first response to the first stimulus from the user at a second point in time, the first response being detected using the respiratory therapy system;
determine a first score based at least in part on (i) the first point in time and (ii) the second point in time;
cause the respiratory therapy device to deliver the supplied pressurized air to the user during a first therapy session;
cause a second test to begin, the second test including causing a second stimulus to be generated at a third point in time;
receive a second response to the second stimulus from the user at a fourth point in time, the second response being detected using the respiratory therapy system;
determine a second score based at least in part on (i) the third point in time and (ii) the fourth point in time; and
communicate a result associated with the first score and the second score to the user.

153. The system of claim 152, wherein the control system is further configured to execute the machine-readable instructions to:
determine the first score based at least in part on an elapsed time between the first point in time and the second point in time; and
determine the second score based at least in part on an elapsed time between the third point in time and the fourth point in time.

154. The system of claim 152, wherein the control system is further configured to execute the machine-readable instructions to:
determine the first point in time for causing the first stimulus to be generated or the second point in time for causing the second stimulus to be generated based at least in part on (i) detecting that the user has donned the user interface, (ii) detecting that a sleep state of the user has transitioned to a wakefulness sleep state, (iii) a current time of day, or (iv) an input from the user.

155-159. (canceled)

160. The system of claim 152, wherein the control system is further configured to execute the machine-readable instructions to:
based at least in part on the change between the first score and the second score, cause an adjustment to (i) a pressure setting of the supplied pressurized air, (ii) humidity setting of the supplied pressurized air, or (iii) both.

161-168. (canceled)
